(19) United States
(12) Patent Application Publication
(10) Pub. No.: US 2010/0113827 A1
(43) Pub. Date: May 6, 2010

(54) Removing iodobenzene compounds from acetic acid

(76) Inventor: Wei Wang, Boothwyn, PA (US)

Correspondence Address: LyondellBasell Industries, 3801 WEST CHESTER PIKE, NEWTOWN SQUARE, PA, 19073, US

(21) Appl. No.: 12/290,757

(22) Filed: Nov. 3, 2008

(51) Int. Cl. C07C 53/08

(52) U.S. Cl. 562/519

(57) ABSTRACT

A method for removing iodobenzene compounds from an acetic acid is disclosed. The method comprises contacting an acetic acid with palladium supported on macroreticular ion-exchange resins. A process for producing acetic acid is also disclosed. The process comprises carbonylating methanol in the presence of a carbonylation catalyst and a triphenylphosphine oxide stabilizer to produce acetic acid which contains iodobenzene compounds and removing the iodobenzene compounds by contacting the acetic acid product with palladium.

### FIELD OF THE INVENTION

The invention relates to the preparation of acetic acid. More particularly, the invention relates to a method for removing iodobenzene compounds from acetic acid.

### BACKGROUND OF THE INVENTION

The carbonylation of methanol produces acetic acid:

CH₃OH+CO→CH₃COOH

Prior to 1970, acetic acid was made using a cobalt catalyst. A rhodium carbonyl iodide catalyst was developed in 1970 by Monsanto. The rhodium catalyst is considerably more active than the cobalt catalyst, which allows lower reaction pressure and temperature. Most importantly, the rhodium catalyst gives high selectivity to acetic acid.

One problem associated with the original Monsanto process is that a large amount of water (about 14%) is needed to produce hydrogen in the reactor via the water-gas shift reaction (CO+H₂O [symbol] CO₂+H₂). Water and hydrogen are needed to react with precipitated Rh(III) and inactive [Rh₄(CO)₂] to regenerate the active Rh(I) catalyst. This large amount of water increases the amount of hydrogen iodide, which is highly corrosive and leads to engineering problems. Further, removing a large amount of water from the acetic acid product is costly.

In the late '70s Celanese modified the carbonylation process by adding lithium iodide salt to the carbonylation. Lithium iodide salt increases the catalyst stability by minimizing the side reactions that produce inactive Rh(III) species and therefore the amount of water needed is reduced. However, the high concentration of lithium iodide salt promotes stress crack corrosion of the reactor vessels. Furthermore, the use of iodide salts increases the iodide impurities in the acetic acid product.

In the early '90s, Millennium Petrochemicals developed a new rhodium carbonylation catalyst system that does not use iodide salt. The catalyst system uses a pentavalent Group VA oxide such as triphenylphosphine oxide as a catalyst stabilizer. The Millennium catalyst system not only reduces the amount of water needed but also increases the carbonylation rate and acetic acid yield. See U.S. Pat. No. 5,817,869.

One challenge still facing the industry is removing iodide compounds from acetic acid. Acetic acid is used to make vinyl acetate by acetoxylation in the presence of palladium-gold catalysts. Iodide compounds inactivate palladium-gold catalysts. Methods for removing iodide compounds from acetic acid are known. For instance, U.S. Pat. No. 5,344,976 teaches how to remove alkyl iodide by silver-exchanged resins. However, silver-exchanged resins are not effective in removing aromatic iodide compounds such as iodobenzene. Aromatic iodide compounds are removed from acetic acid by palladium-gold acetoxylation catalysts. Those catalysts are expensive.

A new method for removing aromatic iodide compounds from acetic acid is needed. Ideally, the method can be more effective and less expensive than palladium-gold catalysts which are currently used in the industry.

### SUMMARY OF THE INVENTION

The invention is a method for removing iodobenzene compounds from acetic acid. The method comprises contacting the acetic acid with palladium supported on a macroreticular ion-exchange resin. The invention also includes a process for producing acetic acid. The process comprises reacting methanol and carbon monoxide in the presence of a carbonylation catalyst, a triphenylphosphine oxide stabilizer, methyl iodide, water and methyl acetate to produce an acetic acid stream containing an iodobenzene compound and flashing at least a portion of the acetic acid stream into a vapor stream comprising acetic acid, water, methanol, methyl acetate, methyl iodide and the iodobenzene compound, and a liquid stream comprising the catalyst and the catalyst stabilizer. The liquid stream is recycled to the carbonylation and the vapor stream is distilled to produce an acetic acid product stream comprising acetic acid and the iodobenzene compound. The acetic acid product stream is contacted with palladium supported on a macroreticular ion-exchange resin to remove the iodobenzene.

### DETAILED DESCRIPTION OF THE INVENTION

The invention is a method for removing iodobenzene compounds from acetic acid. The method comprises contacting the acetic acid with palladium supported on a macroreticular ion-exchange resin. Macroreticular ion-exchange resins are known in the art and they comprise two continuous phases, i.e. a continuous pore phase and a continuous polymeric phase. The polymeric phase is structurally composed of small spherical microgel particles agglomerated together to form clusters, which, in turn are fastened together at their interphases and form interconnecting pores. Typically, macroreticular ion-exchange resins have a specific surface area in the range of from 5 to 1500 m²/g. The surface area arises from the freely exposed surface of the microgel particles. Macroreticular ion-exchange resins typically have an average pore diameter in the range of from 1 to 1000 nm, usually of from 10 to 100 nm. Macroreticular ion-exchange resins are to be contrasted with gel-type resins, which do not have permanent pore structures. Methods for making macroreticular ion-exchange resins are known. See U.S. Pat. No. 7,098,252, teachings of which are herein incorporated by reference.

Palladium can be supported on the macroreticular ion-exchange resin by any known method. In one method, the macroreticular resin is preferably impregnated with a palladium salt. Preferably, the impregnation is performed in aqueous solutions. Suitable palladium salts include palladium chloride, sodium chloropalladite, palladium nitrate, palladium sulfate, palladium tetraamine dinitrate, the like, and mixtures thereof. The palladium-impregnated macroreticular resin is reduced to convert the palladium salts to its metal state. The reduction is performed by heating in the presence of a reducing agent. Suitable reducing agents include ammonia, carbon monoxide, hydrogen, hydrocarbons, olefins, aldehydes, alcohols, hydrazine, primary amines, carboxylic acids, carboxylic acid salts, carboxylic acid esters, the like, and mixtures thereof. Hydrogen, ethylene, propylene, alkaline hydrazine, alkaline formaldehyde, and formic acid are preferred reducing agents and ethylene, hydrogen and formic acid are particularly preferred.

Iodobenzene compounds are produced by the side reactions of methanol carbonylation. Examples of iodobenzene compounds include iodobenzene, C₁-C₆ alkyl substituted iodobenzenes, the like, and mixtures thereof. Alkyl substituted iodobenzenes commonly seen in the methanol carbonylation are iodotoluene, iodoethylbenzene, iodoxylenes, the like, and mixtures thereof. The carbonylation reaction is usually performed in the presence of a carbonylation catalyst and a catalyst stabilizer. Suitable carbonylation catalysts include those known in the acetic acid industry. Examples of suitable carbonylation catalysts include rhodium catalysts and iridium catalysts.

Suitable rhodium catalysts are taught, for example, by U.S. Pat. No. 5,817,869. Suitable rhodium catalysts include rhodium metal and rhodium compounds. Preferably, the rhodium compounds are selected from the group consisting of rhodium salts, rhodium oxides, rhodium acetates, organo-rhodium compounds, coordination compounds of rhodium, the like, and mixtures thereof. More preferably, the rhodium compounds are selected from the group consisting of Rh₂(CO)₄I₂, Rh₂(CO)₄Br₂, Rh₂(CO)₄Cl₂, Rh(CH₃CO₂)₂, Rh(CH₃CO₂)₃, [H]Rh(CO)₂I₂, the like, and mixtures thereof. Most preferably, the rhodium compounds are selected from the group consisting of [H]Rh(CO)₂I₂, Rh(CH₃CO₂)₂, the like, and mixtures thereof.

Suitable iridium catalysts are taught, for example, by U.S. Pat. No. 5,932,764. Suitable iridium catalysts include iridium metal and iridium compounds. Examples of suitable iridium compounds include IrCl₃, IrI₃, IrBr₃, [Ir(CO)₂I]₂, [Ir(CO)₂Cl]₂, [Ir(CO)₂Br]₂, [Ir(CO)₄I₂]⁻H⁺, [Ir(CO)₂Br₂]⁻H⁺, [Ir(CO)₂I₂]⁻H⁺, [Ir(CH₃)I₃(CO)₂]⁻H⁺, Ir₄(CO)₁₂, IrCl₃.4H₂O, IrBr₃.4H₂O, Ir₃(CO)₁₂, Ir₂O₃, IrO₂, Ir(acac)(CO)₂, Ir(acac)₃, Ir(OAc)₃, [Ir₃O(OAc)₆(H₂O)₃][OAc], and H₂[IrCl₆]. Preferably, the iridium compounds are selected from the group consisting of acetates, oxalates, acetoacetates, the like, and mixtures thereof. More preferably, the iridium compounds are acetates.

The iridium catalyst is preferably used with a co-catalyst. Preferred co-catalysts include metals and metal compounds selected from the group consisting of osmium, rhenium, ruthenium, cadmium, mercury, zinc, gallium, indium, and tungsten, their compounds, the like, and mixtures thereof. More preferred co-catalysts are selected from the group consisting of ruthenium compounds and osmium compounds. Most preferred co-catalysts are ruthenium compounds. Preferably, the co-catalysts are acetates.

The carbonylation reaction is preferably performed in the presence of a catalyst stabilizer. Suitable catalyst stabilizers include those known to the industry. In general, there are two types of catalyst stabilizers. The first type of catalyst stabilizer is metal iodide salt such as lithium iodide. The second type of catalyst stabilizer is a non-salt stabilizer. Preferred non-salt stabilizers are pentavalent Group VA oxides. See U.S. Pat. No. 5,817,869. Phosphine oxides are more preferred. Triphenylphosphine oxides are most preferred.

The carbonylation reaction is preferably performed in the presence of water. Preferably, the concentration of water present is from about 2 wt % to about 14 wt % based on the total weight of the reaction medium. More preferably, the water concentration is from about 2 wt % to about 10 wt %. Most preferably, the water concentration is from about 4 wt % to about 8 wt %.

The reaction is preferably performed in the presence of methyl acetate. Methyl acetate can be formed in situ. If desirable, methyl acetate can be added as a starting material to the reaction mixture. Preferably, the concentration of methyl acetate is from about 2 wt % to about 20 wt % based on the total weight of the reaction medium. More preferably, the concentration of methyl acetate is from about 2 wt % to about 16 wt %. Most preferably, the concentration of methyl acetate is from about 2 wt % to about 8 wt %. Alternatively, methyl acetate or a mixture of methyl acetate and methanol from byproduct streams of the hydroysis/methanolysis of polyvinyl acetate can be used for the carbonylation reaction.

The reaction is usually performed in the presence of methyl iodide. Methyl iodide is a catalyst promoter. Preferably, the concentration of methyl iodide is from about 0.6 wt % to about 36 wt % based on the total weight of the reaction medium. More preferably, the concentration of methyl iodide is from about 4 wt % to about 24 wt %. Most preferably, the concentration of methyl iodide is from about 6 wt % to about 20 wt %. Alternatively, methyl iodide can be generated in the carbonylation reactor by adding hydrogen iodide (HI).

Methanol and carbon monoxide are fed to the carbonylation reactor. The methanol feed to the carbonylation reaction can come from a syngas-methanol facility or any other source. Methanol does not react directly with carbon monoxide to form acetic acid. It is converted to methyl iodide by the hydrogen iodide present in the reactor and then reacts with carbon monoxide and water to give acetic acid and regenerate hydrogen iodide. Carbon monoxide not only becomes part of the acetic acid molecule, but it also plays an important role in the formation and stability of the active catalyst.

The carbonylation reaction is preferably performed at a temperature within the range of about 150° C. to about 250° C. More preferably, the reaction is performed at a temperature within the range of about 150° C. to about 200° C. The carbonylation reaction is preferably performed under a pressure within the range of about 200 psig to about 1,000 psig. More preferably, the reaction is performed under a pressure within the range of about 300 psig to about 500 psig.

An acetic acid product stream is preferably withdrawn from the reactor and is separated, by a flash separation, into a liquid fraction comprising the catalyst and the catalyst stabilizer and a vapor fraction comprising the acetic acid product, the reactants, water, methyl iodide, and impurities generated during the carbonylation reaction including iodobenzene compounds. The liquid fraction is preferably recycled to the carbonylation reactor. The vapor fraction is preferably then passed to the so-called “light ends distillation” and separated into an overhead stream comprising methyl iodide, water, methanol, and methyl acetate, and an acetic acid stream comprising acetic acid, a small amount of water, some heavy impurities such as propionic acid, and the iodobenzene compounds. The acetic acid stream is preferably passed to a drying column to remove water and then be subjected to the so-called “heavy ends distillation” to remove the heavy impurities. The acetic acid stream from the heavy ends distillation passes through the macroreticular ion-exchange resin supported palladium bed to remove the iodobenzene compounds. Optionally, prior to passing the macroreticular ion-exchange resin supported palladium bed, the acetic acid stream from the heavy ends distillation passes a silver-exchanged resin to remove alkyl iodides.

The following examples are merely illustrative. Those skilled in the art will recognize many variations that are within the spirit of the invention and scope of the claims.

### EXAMPLE 1

### Removal of Iodobenzene from Acetic Acid with Palladium Supported on Macroreticular Ion-Exchange Resin

A macroreticular ion-exchange resin, Amberlyst 15 (wet form, 50 grams, and a product of Rohm & Haas) and deionized water (100 ml) are mixed in a 500-ml flask. A diluted palladium tetraamine dinitrate solution is prepared by mixing 4.6 grams of Pd tetraamine dinitrate solution (product of Engelhand, 5.4% Pd) with 20 ml of DI water. The diluted palladium nitrate solution is dropwisely added into the flask containing Amberlyst 15/water mixture under stirring at room temperature (25° C.). The stirring is continued for two hours after addition. The Pd-loaded Amberlyst 15 resin is isolated from reaction solution and washed with deionized water, and then reduced by the following procedure. Amberlyst 15-Pd resin is added into a 1-liter flask containing 500 grams of 10% aqueous formic acid solution. The reaction content is refluxed for 2 hours. After reaction, the reduced Pd-Amberlyst 15 is collected and stocked under the protection of 1% formic acid solution.

Pd-Amberlyst 15 prepared above (2.0 grams) is loaded into a jacketed, adjustable bed column which is connected with a thermostat. The temperature of the jacked column is set at 45° C. An acetic acid sample containing 20 ppb of iodobenzene is pumped through the column at 0.25, 0.5, 0.75, 1.0 and 1.25 ml/min respectively. The acetic acid sample collected from the bottom of the column is tested by GC using ECD detector to determine the iodobenzene contents. The results are summarized in Table 1.

### EXAMPLE 2

### Removal of Iodobenzene from Acetic Acid with Palladium Supported on Macroreticular Ion-Exchange Resin

The general procedure of Example 1 is repeated but Amberlyst 15 is replaced by Purolite 145 (a product of Purolite Inc.). The results are summarized in Table 1.

### COMPARATIVE EXAMPLE 3

### Removal of Iodobenzene from Acetic Acid with Palladium Supported on Gel-Type Ion-Exchange Resin

The general procedure of Example 1 is repeated but Amberlyst 15 is replaced by a gel-type ion-exchange resin, SGC650 (a product of Dow Chemical Company). The results are summarized in Table 1.

### COMPARATIVE EXAMPLE 4

### Removal of Iodobenzene from Acetic Acid with Palladium Supported on Activated Carbon

The general procedure of Example 1 is repeated but Amberlyst 15 is replaced by an activated carbon from Aldrich. The results are summarized in Table 1.

### COMPARATIVE EXAMPLE 5

### Removal of Iodobenzene from Acetic Acid with Palladium-Gold Supported on Aluminum Silicate

The general procedure of Example 1 is repeated but a Pd—Au catalyst supported on alumino silicate, H5250 (product of Engelhard Corporation) is used. The results are summarized in Table 1.

The results in Table 1 indicate that palladium supported on macroreticular ion-exchange resins are significantly more effective in removing iodobenzene than palladium supported on a gel-type ion-exchange resin or activated carbon.

The results in Table 1 also indicate that palladium supported on macroreticular ion-exchange resins is significantly more effective in removing iodobenzene than palladium-gold supported on aluminosilicate.

TABLE 1
RESULTS SUMMARY

| | Iodobenzene Removal (%) | | | | |
|---|---|---|---|---|---|
| | Ex. No. | | | | |
| Flow rate | 1 | 2 | C3 | C4 | C5 Pd—Au- |
| (ml/min) | Pd-Amberlyst 15 | Pd-Purolite 145 | Pd-SGC650 | Pd-Activated C | Silica |
| 0.25 | 100 | 100 | 43 | 12 | 84 |
| 0.5 | 96 | 100 | 18 | 11 | 78 |
| 0.75 | 94 | 99 | 18 | 7.6 | 71 |
| 1.0 | 92 | 100 | 18 | 8.5 | 71 |
| 1.25 | 91 | 99 | 15 | 7.2 | 68 |
| 1.5 | 91 | 99 | 18 | 6.5 | 70 |

### COMPARATIVE EXAMPLE 6

### Removal of Iodobenzene from Acetic Acid with Silver Supported on Macroreticular Ion-Exchange Resin

A macroreticular ion-exchange resin, Amberlyst 15 (wet form, 45 grams, and a product of Rohm & Haas) and deionized water (150 ml) are mixed in a 500-ml flask. A silver nitrate solution is prepared by mixing 4.25 grams of silver nitrate (product of Aldrich) with 50 ml of deionized water. The silver nitrate solution is dropwisely added into the flask containing Amberlyst 15/water mixture under stirring at room temperature (25° C.). The stirring is continued for two hours after addition. The silver-loaded Amberlyst 15 resin is isolated from reaction solution and washed with deionized water until no fugitive silver is detected.

Silver-Amberlyst 15 prepared above (25 ml) is loaded into a jacketed, adjustable bed column which is connected with a thermostat. The temperature of the jacked column is set at 60° C. An acetic acid sample containing 20.8 ppb of iodobenzene is pumped through the column at 3.3 ml/min. The acetic acid sample collected from the bottom of the column is tested by GC using ECD detector to determine the iodobenzene contents, which is 20.8 ppb.

I claim:

1. A method for removing an iodobenzene compound from acetic acid, said method comprising contacting the acetic acid with palladium supported on a macroreticular ion-exchange resin.

2. The method of claim 1, wherein the iodobenzene compound is iodobenzene.

3. The method of claim 1, wherein the iodobenzene compound is a C₁-C₆ alkyl substituted iodobenzene.

4. The method of claim 1, wherein the acetic acid is produced by carbonylating methanol in the presence of a carbonylation catalyst and a triphenylphosphine oxide stabilizer.

5. A process for producing acetic acid, said process comprising:
(a) reacting methanol and carbon monoxide in the presence of a carbonylation catalyst, a triphenylphosphine oxide stabilizer, methyl iodide, water and methyl acetate to produce an acetic acid stream containing an iodobenzene compound;
(b) flashing at least a portion of the acetic acid stream into a vapor stream comprising acetic acid, water, methanol, methyl acetate, methyl iodide and the iodobenzene compound, and a liquid stream comprising the catalyst and the catalyst stabilizer;
(c) subjecting the vapor stream to a distillation to produce an acetic acid product stream comprising acetic acid and the iodobenzene compound; and
(d) contacting the acetic acid product stream with palladium supported on a macroreticular ion-exchange resin to remove the iodobenzene.

6. The process of claim 5, wherein the iodobenzene compound is iodobenzene.

7. The process of claim 5, wherein the iodobenzene compound is a C₁-C₆ alkyl substituted iodobenzene.

8. The process of claim 5, wherein the liquid stream from step (b) is recycled to the carbonylation step (a).

9. The process of claim 5, wherein the distillation in step (c) is a light ends distillation.
